(19) United States
(12) Patent Application Publication
(10) Pub. No.: US 2014/0275280 A1
(43) Pub. Date: Sep. 18, 2014

(54) Therapeutic uses of geranylgeranyl acetone and derivatives thereof

(71) Applicant: COYOTE PHARMACEUTICALS, INC., MENLO PARK, CA (US)

(72) Inventors: Hiroaki Serizawa, Menlo Park, CA (US); Tilmann M. Brotz, Menlo Park, CA (US)

(21) Appl. No.: 13/815,740

(22) Filed: Mar. 15, 2013

(51) Int. Cl. A61K 31/121

(52) U.S. Cl. CPC A61K 31/121
USPC 514/675

(57) ABSTRACT

Provide herein are methods of treating inflammatory bowel disease with geranylgeranyl acetone (GGA) and/or derivatives thereof. Also provided are methods of treating chronic liver disease (CLD) with geranylgeranyl acetone (GGA) and/or derivatives thereof. Still further are provided methods for treating other hepatic and cardiac disorders.

### FIELD OF THE INVENTION

This invention provides therapeutic methods for treating inflammatory bowel disease (IBD) or a related disorder or condition by the administration of compositions that include geranylgeranyl acetone (GGA) and derivatives thereof. This invention also provides therapeutic methods for treating chronic liver disease (CLD) or a related disorder or condition or acute liver injury or failure by the administration of compositions that include GGA and derivatives thereof. Furthermore this invention provides therapeutic methods for treating cardiac ischemia and repurfusion injury or a related disorder or condition by the administration of compositions that include GGA and derivatives thereof. Preferably, GGA or the GGA derivative is enriched in the all trans isomer, compared to the relative amount of the trans isomer in the mixtures of cis and trans isomers of GGA or the GGA derivative.

### STATE OF THE ART

Geranylgeranyl acetone (GGA) has the formula:

[chemical structure]

See, for example, PCT Pat. App. Pub. No. WO 2012/031028 and PCT Pat. App. No. PCT/US2012/027147, each of which is incorporated herein by reference in its entirety.

Inflammatory bowel disease (IBD) is generally characterized by diarrhea, cramping, abdominal pains, weight loss, rectal bleeding, tiredness, anemia, fistulae, perforations, obstruction of the bowel and frequent need for surgical intervention. It encompasses a number of disorders including Crohn's disease, ulcerative colitis, indeterminate colitis, microscopic colitis and collagenous colitis. Such disorders may at times begin clinically with a more benign or milder presentation, resembling Irritable Bowel Syndrome (IBS) which can subsequently progress to increasing inflammation accompanying the IBS and may ultimately develop full-blown IBD. The precise causes of IBD and IBS remain unknown.

Chronic liver disease (CLD) is marked by the gradual destruction of liver tissue over time. Several liver diseases can fall under this category, including without limitation, cirrhosis and fibrosis, the latter of which is often the precursor to cirrhosis, non-alcoholic fatty liver disease, and non-alcoholic steatohepatitis.

Cirrhosis is the result of acute and chronic liver disease and is characterized by the replacement of liver tissue by fibrotic scar tissue and regenerative nodules leading to a progressive loss of liver function. Fibrosis and nodular regeneration results in the loss of the normal microscopic lobular architecture of the liver. Fibrosis represents the growth of scar tissue resulting from, for example, infection, inflammation, injury, and even healing. Over time, the fibrotic scar tissue slowly replaces the normal functioning liver tissue resulting in a decreasing amount of blood flow to the liver leaving the liver incapable of fully processing nutrients, hormones, drugs, and poisons that are found in the bloodstream. More common causes of cirrhosis include alcoholism, hepatitis C viral infections, ingestion of toxins, and fatty liver, but many other possible causes also exist.

Liver injury is some form of trauma sustained to the liver. This can occur through either a blunt force such as a car accident, or a penetrating foreign object such as a knife. Liver injuries constitute 5% of all traumas, making it the most common abdominal injury.

Acute liver failure is the appearance of severe complications rapidly after the first signs of liver disease (such as jaundice), and indicates that the liver has sustained severe damage (loss of function of 80-90% of liver cells). The complications are hepatic encephalopathy and impaired protein synthesis (as measured by the levels of serum albumin and the prothrombin time in the blood). The 1993 classification defines hyperacute as within 1 week, acute as 8-28 days and subacute as 4-12 weeks. It reflects the fact that the pace of disease evolution strongly influences prognosis. Acetaminophen hepatotoxicity is, by far, the most common cause of acute liver failure in both the United States and the United Kingdom. Toxicity of acetaminophen arises often due to its quinone metabolite. Acetaminophen overdose results in more calls to poison control centers in the US than overdose of any other pharmacological substance. Signs and symptoms of paracetamol toxicity may initially be absent or vague. Untreated overdose can lead to liver failure and death within days. Renal failure is also a possible side effect.

Coronary heart disease (CHD) is the narrowing or blockage of the coronary arteries, usually caused by atherosclerosis. Atherosclerosis (sometimes called “hardening” or “clogging” of the arteries) is the buildup of cholesterol and fatty deposits (called plaques) on the inner walls of the arteries. These plaques can restrict blood flow to the heart muscle by physically clogging the artery or by causing abnormal artery tone and function.

Without an adequate blood supply, the heart becomes starved of oxygen and the vital nutrients it needs to work properly. This can cause chest pain called angina. If blood supply to a portion of the heart muscle is cut off entirely, or if the energy demands of the heart become much greater than its blood supply, a heart attack (injury to the heart muscle) may occur.

Cardiac ischemia may be asymptomatic or may cause chest pain, known as angina pectoris. It occurs when the heart muscle, or myocardium, receives insufficient blood flow. This most frequently results from atherosclerosis, which is the long-term accumulation of cholesterol-rich plaques in the coronary arteries. Ischemic heart disease is the most common cause of death in most Western countries and a major cause of hospital admissions.

### SUMMARY OF THE INVENTION

In one aspect of the invention, a method is provided of treating inflammatory bowel disease (IBD) or a related disorder or condition comprising administering a composition comprising an effective amount of geranylgeranyl acetone (GGA) or a GGA derivative, or a pharmaceutically acceptable salt thereof, and a pharmaceutically acceptable excipient to a subject in need thereof. As used herein, subject or patient refers to a mammal, particularly preferably humans. In another aspect, a method is provided of upregulating HSP70 in stomach cells affected by IBD comprising contacting the stomach cells with an effective amount of GGA.

In another aspect of the invention, a method is provided of treating chronic liver disease or a related disorder or condition comprising administering a composition comprising an effective amount of geranylgeranyl acetone (GGA) or a GGA derivative, or a pharmaceutically acceptable salt thereof, and a pharmaceutically acceptable excipient. In another aspect, a method is provided of upregulating HSP70 in hepatic cells affected by a chronic liver disease comprising contacting the hepatic cells with an effective amount of GGA. In some embodiments, the hepatic cells are affected with cirrhosis, fibrosis, non-alcoholic fatty liver disease, or non-alcoholic steatohepatitis.

In another aspect of the invention, a method is provided of treating a disorder selected from liver injury, preferably acute liver injury (from trauma, surgery or as a side effect of cancer treatment), acute liver failure, preferably caused by drug toxicity such as acetaminophen toxicity, cardiac ischemia, myocardial infarction, repurfusion injury and heart transplants, or a related disorder or condition, comprising administering a composition comprising an effective amount of geranylgeranyl acetone (GGA) or a GGA derivative, or a pharmaceutically acceptable salt thereof, and a pharmaceutically acceptable excipient, to a subject in need thereof. In some embodiments the (GGA) or a GGA derivative is administered to the subject in an emergency room setting.

In another aspect of the invention, a method is provided of treating a subject diagnosed with mild to moderate IBD following gastrectomy comprising administering a composition comprising an effective amount of geranylgeranyl acetone (GGA) or a GGA derivative, or a pharmaceutically acceptable salt thereof, and a pharmaceutically acceptable excipient. In a preferred embodiment, the subjects are treated for 12 weeks.

Preferably, the GGA or the GGA derivative includes the all-trans (hereinafter “trans”) form or substantially the trans form of the GGA or the GGA derivative. As used herein, “substantially” in the context of cis/trans configurations refers to at least 80%, more preferably at least 90%, yet more preferably at least 95%, and most preferably at least 99% of the desired configuration, which can include at least 80%, more preferably at least 90%, yet more preferably at least 95%, and most preferably at least 99% of the trans isomer. In certain preferred embodiments of the invention, the GGA or a GGA derivative exists at least 80%, or at least 90%, or at least 95%, or at least 99% in the trans isomer.

In certain aspects, this invention relates to pharmaceutical uses of geranylgeranyl acetone (GGA) and GGA derivatives, pharmaceutical compositions of isomers of geranylgeranyl acetone, preferably synthetic geranylgeranyl acetone, and GGA derivatives, and methods of using such compounds and pharmaceutical compositions. In certain aspects, this invention relates to a 5-trans isomer compound of formula VI:

[chemical structure]

wherein VI is at least 80% in the 5E, 9E, 13E configuration. In one embodiment, this invention utilizes a compound, which is synthetic 5E, 9E, 13E geranylgeranyl acetone. In another embodiment, the synthetic 5E, 9E, 13E geranylgeranyl acetone is free of 5Z, 9E, 13E geranylgeranyl acetone. In another aspect, this invention provides a pharmaceutical composition comprising synthetic GGA or synthetic 5E, 9E, 13E GGA, and at least one pharmaceutical excipient.

Another aspect of this invention relates to a synthetic 5-cis isomer compound of formula VII:

[chemical structure]

wherein VII is at least 80% in the 5Z, 9E, 13E configuration, or a ketal thereof of formula XII:

[chemical structure]

wherein each R₇₀ independently is C₁-C₆ alkyl, or two R₇₀ groups together with the oxygen atoms they are attached to form a 5 or 6 membered ring, which ring is optionally substituted with 1-3, preferably 1-2, C₁-C₆ alkyl groups. Preferably, the two R₇₀ groups are the same. In one embodiment, R₇₀ is, methyl, ethyl, or propyl. In another embodiment, the cyclic ring is:

[chemical structure]

### DETAILED DESCRIPTION OF THE INVENTION

### Definitions

It must be noted that as used herein and in the appended claims, the singular forms “a”, “an”, and “the” include plural referents unless the context clearly dictates otherwise. Thus, for example, reference to “a solvent” includes a plurality of such solvents.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition or process consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations. Each numerical parameter should at least be construed in light of the number of reported significant digits and by applying ordinary rounding techniques.

As used herein, Cₘ-Cₙ, such as C₁-C₁₀, C₁-C₆, or C₁-C₄ when used before a group refers to that group containing m to n carbon atoms.

The term “about” when used before a numerical designation, e.g., temperature, time, amount, and concentration, including range, indicates approximations which may vary by (+) or (−) 10%, 5% or 1%.

The term “alkoxy” refers to —O-alkyl.

The term “alkyl” refers to monovalent saturated aliphatic hydrocarbyl groups having from 1 to 10 carbon atoms (i.e., C₁-C₁₀ alkyl) or 1 to 6 carbon atoms (i.e., C₁-C₆ alkyl), or 1 to 4 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “aryl” refers to a monovalent, aromatic mono- or bicyclic ring having 6-10 ring carbon atoms. Examples of aryl include phenyl and naphthyl. The condensed ring may or may not be aromatic provided that the point of attachment is at an aromatic carbon atom. For example, and without limitation, the following is an aryl group:

[chemical structure]

The term “—CO₂H ester” refers to an ester formed between the —CO₂H group and an alcohol, preferably an aliphatic alcohol. A preferred example included —CO₂R^{E}, wherein R^{E} is alkyl or aryl group optionally substituted with an amino group.

The term “chiral moiety” refers to a moiety that is chiral. Such a moiety can possess one or more asymmetric centers. Preferably, the chiral moiety is enantiomerically enriched, and more preferably a single enantiomer. Non limiting examples of chiral moieties include chiral carboxylic acids, chiral amines, chiral amino acids, such as the naturally occurring amino acids, chiral alcohols including chiral steroids, and the likes.

The term “cycloalkyl” refers to a monovalent, preferably saturated, hydrocarbyl mono-, bi-, or tricyclic ring having 3-12 ring carbon atoms. While cycloalkyl, refers preferably to saturated hydrocarbyl rings, as used herein, it also includes rings containing 1-2 carbon-carbon double bonds. Nonlimiting examples of cycloalkyl include cyclopropyl, cyclobutyl, cyclopentyl, cyclohexyl, cycloheptyl, adamentyl, and the like. The condensed rings may or may not be non-aromatic hydrocarbyl rings provided that the point of attachment is at a cycloalkyl carbon atom. For example, and without limitation, the following is a cycloalkyl group:

[chemical structure]

The term “halo” refers to F, Cl, Br, and/or I.

The term “heteroaryl” refers to a monovalent, aromatic mono-, bi-, or tricyclic ring having 2-14 ring carbon atoms and 1-6 ring heteroatoms selected preferably from N, O, S, and P and oxidized forms of N, S, and P, provided that the ring contains at least 5 ring atoms. Nonlimiting examples of heteroaryl include furan, imidazole, oxadiazole, oxazole, pyridine, quinoline, and the like. The condensed rings may or may not be a heteroatom containing aromatic ring provided that the point of attachment is a heteroaryl atom. For example, and without limitation, the following is a heteroaryl group:

[chemical structure]

The term “heterocyclyl” or heterocycle refers to a non-aromatic, mono-, bi-, or tricyclic ring containing 2-10 ring carbon atoms and 1-6 ring heteroatoms selected preferably from N, O, S, and P and oxidized forms of N, S, and P, provided that the ring contains at least 3 ring atoms. While heterocyclyl preferably refers to saturated ring systems, it also includes ring systems containing 1-3 double bonds, provided that they ring is non-aromatic. Nonlimiting examples of heterocyclyl include, azalactones, oxazoline, piperidinyl, piperazinyl, pyrrolidinyl, tetrahydrofuranyl, and tetrahydropyranyl. The condensed rings may or may not contain a non-aromatic heteroatom containing ring provided that the point of attachment is a heterocyclyl group. For example, and without limitation, the following is a heterocyclyl group:

[chemical structure]

The term “hydrolyzing” refers to breaking an R^{H}—O—CO—, R^{H}—O—CS—, or an R^{H}—O—SO₂— moiety to an R^{H}—OH, preferably by adding water across the broken bond. A hydrolyzing is performed using various methods well known to the skilled artisan, non limiting examples of which include acidic and basic hydrolysis.

The term “oxo” refers to a C═O group, and to a substitution of 2 geminal hydrogen atoms with a C═O group.

The term “pharmaceutically acceptable” refers to safe and non-toxic for in vivo, preferably, human administration.

The term “pharmaceutically acceptable salt” refers to a salt that is pharmaceutically acceptable.

The term “salt” refers to an ionic compound formed between an acid and a base. When the compound provided herein contains an acidic functionality, such salts include, without limitation, alkai metal, alkaline earth metal, and ammonium salts. As used herein, ammonium salts include, salts containing protonated nitrogen bases and alkylated nitrogen bases. Exemplary, and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionally, such salts include, without limitation, salts of organic acids, such as caroboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisalfate, mono-, di-, and tribasic phosphate, mesylate, tosylate, and the likes.

The term “substantially pure trans isomer” refers to a trans isomer that is by molar amount 95%, preferably 96%, more preferably 99%, and still more preferably 99.5% or more a trans isomer with the rest being the corresponding cis isomer.

“Trans” in the context of GGA and GGA derivatives refer to the GGA scaffold as illustrated below:

[chemical structure]

wherein R¹-R⁵ is defined herein and q is 0-2. As shown, each double bond is in a trans or E configuration. In contrast, a cis form of GGA or a GGA derivative will contain one or more of these bonds in a cis or Z configuration.

As used herein, the term “intranuclear” or “intranuclearly” refers to the space inside the nuclear compartment of an animal cell.

The term “cytoplasm” refers to the space outside of the nucleus but within the outer cell wall of an animal cell.

The term “G protein” refers to a family of proteins involved in transmitting chemical signals outside the cell and causing changes inside of the cell. The Rho family of G proteins is small G protein, which are involved in regulating actin cytoskeletal dynamics, cell movement, motility, transcription, cell survival, and cell growth. RHOA, RAC1, and CDC42 are the most studied proteins of the Rho family. Active G proteins are localized to the cellular membrane where they exert their maximal biological effectiveness.

The terms “treat”, “treating” or “treatment”, as used herein, include alleviating, abating or ameliorating a disease or condition or one or more symptoms thereof, preventing additional symptoms, ameliorating or preventing the underlying metabolic causes of symptoms, inhibiting the disease or condition, e.g., arresting or suppressing the development of the disease or condition, relieving the disease or condition, causing regression of the disease or condition, relieving a condition caused by the disease or condition, or suppressing the symptoms of the disease or condition, and are intended to include prophylaxis. The terms also include relieving the disease or conditions, e.g., causing the regression of clinical symptoms. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the individual, notwithstanding that the individual is still be afflicted with the underlying disorder. For prophylactic benefit, the compositions are administered to an individual at risk of developing a particular disease, or to an individual reporting one or more of the physiological symptoms of a disease, even though a diagnosis of this disease has not been made.

The terms “preventing” or “prevention” refer to a reduction in risk of acquiring a disease or disorder (i.e., causing at least one of the clinical symptoms of the disease not to develop in a subject that may be exposed to or predisposed to the disease but does not yet experience or display symptoms of the disease). The terms further include causing the clinical symptoms not to develop, for example in a subject at risk of suffering from such a disease or disorder, thereby substantially averting onset of the disease or disorder.

The term “effective amount” refers to an amount that is effective for the treatment of a condition or disorder by an administration of a compound or composition described herein. In some embodiments, an effective amount of any of the compositions or dosage forms described herein is the amount used to treat inflammatory bowel disease or a related disorder or condition and/or to reduce one or more negative effects of inflammatory bowel disease or a related disorder or condition comprising administering any of the compositions or dosage forms described herein to a subject in need thereof.

The term “carrier” as used herein, refers to relatively nontoxic chemical compounds or agents that facilitate the incorporation of a compound into cells or tissues.

### Compounds:

### GGA

This invention relates to compounds and pharmaceutical compositions of isomers of geranylgeranyl acetone. In certain aspects, this invention relates to a synthetic 5-trans isomer compound of formula VI:

[chemical structure]

wherein VI is at least 80% in the 5E, 9E, 13E configuration. In some embodiments, the invention provides for a compound of formula VI wherein VI is at least 85%, or at least 90%, or at least 95%, or at least 96%, or at least 97%, or at least 98%, or at least 99%, or at least 99.5%, or at least 99.9% in the 5E, 9E, 13E configuration. In some embodiments the invention for the compound of formula VI does not contain any of the cis-isomer of GGA.

Another aspect of this invention relates to a synthetic 5-cis isomer compound of formula VII:

[chemical structure]

wherein VII is at least 75% in the 5Z, 9E, 13E configuration. In certain embodiments, the invention provides for a compound of formula VII wherein VII is at least 80% in the 5E, 9E, 13E configuration, or alternatively, at least 85%, or at least 90%, or at least 95%, or at least 96%, or at least 97%, or at least 98%, or at least 99%, or at least 99.5%, or at least 99.9% in the 5E, 9E, 13E configuration. In some embodiments of the invention, the compound of formula VII does not contain any of the trans-isomer of GGA.

The configuration of compounds can be determined by methods known to those skilled in the art such as chiroptical spectroscopy and nuclear magnetic resonance spectroscopy.

The data contained in the examples herewith demonstrate at low concentrations the trans-isomer of GGA is pharmacologically active and shows a dose-dependent relationship. In contrast, the cis-isomer of GGA does not demonstrate a dose dependent relationship and is deemed to be at best of minimal activity.

### GGA Derivatives

GGA derivatives useful in this invention include those described in PCT publication no. WO 2012/031028 and PCT application no. PCT/US2012/027147, each of which are incorporated herein by reference in its entirety. These and other GGA derivatives provided and/or utilized herein are structurally shown below.

In one aspect, the GGA derivative provided and/or utilized herein is of Formula I:

[chemical structure]

or a tautomer or pharmaceutically acceptable salt thereof, wherein
n¹ is 1 or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently are hydrogen or C₁-C₆ alkyl;

### Q¹ is —(C═O)—, —(C═S)—; or —S(O₂)—;

Q₂ is hydrogen, R⁶, —O—R⁶, —NR⁷R⁸, or is a chiral moiety;

### R⁶ is:

C₁-C₆ alkyl, optionally substituted with —CO₂H or an ester thereof, C₁-C₆ alkoxy, oxo, —OH, —CR═CR₂, —C≡CR, C₃-C₁₀ cycloalkyl, C₃-C₈ heterocyclyl, C₆-C₁₀ aryl, C₂-C₁₀ heteroaryl, wherein each R independently is hydrogen or C₁-C₆ alkyl;

CO—C₁-C₆ alkyl;

C₃-C₁₀ cycloalkyl;

C₃-C₈ heterocyclyl;

C₆-C₁₀ aryl; or

C₂-C₁₀ heteroaryl;

wherein each cycloalkyl, heterocyclyl, aryl, or heteroaryl is optionally substituted with 1-3 alkyl groups; —CF₃, 1-3 halo, preferably, chloro or fluoro, groups; 1-3 nitro groups; 1-3 C₁-C₆ alkoxy groups; —CO-phenyl; or —NR¹⁸R¹⁹, each R¹⁸ and R¹⁹ independently is hydrogen; C₁-C₆ alkyl, optionally substituted with —CO₂H or an ester thereof, C₁-C₆ alkoxy, oxo, —CR═CR₂, —CCR, C₃-C₁₀ preferably C₃-C₈ cycloalkyl, C₃-C₈ heterocyclyl, C₆-C₁₀ aryl, or C₂-C₁₀ heteroaryl, wherein each R independently is hydrogen or C₁-C₆ alkyl; C₃-C₁₀ cycloalkyl; C₃-C₈ heterocyclyl; C₆-C₁₀ aryl; or C₂-C₁₀ heteroaryl; wherein each cycloalkyl, heterocyclyl, aryl, or heteroaryl is optionally substituted with 1-3 alkyl groups, optionally substituted with 1-3 halo, preferably, fluoro, groups, where R¹⁸ and R¹⁹ together with the nitrogen atom they are attached to form a 5-7 membered heterocycle;
each R⁷ and R⁸ are independently hydrogen or defined as R⁶; and [symbol]
refers to a mixture of cis and trans isomers at the corresponding position wherein at least 80% and, preferably, no more than 95% of the compound of Formula (I) is present as a trans isomer.

In one embodiment, the GGA derivative provided and/or utilized is of Formula (I-A):

[chemical structure]

as a substantially pure trans isomer around the 2,3 double bond wherein, n¹, R¹-R⁵, Q¹, and Q² are defined as in Formula (I) above.

In another embodiment, n¹ is 1. In another embodiment, n¹ is 2.

In another embodiment, the GGA derivative provided and/or utilized is of Formula (I-B):

[chemical structure]

as a substantially pure trans isomer around the 2,3 double bond wherein, R¹-R⁵, Q¹, and Q² are defined as in Formula (I) above.

In another embodiment, the GGA derivative provided and/or utilized is of Formula I-C:

[chemical structure]

wherein Q¹ and Q² are defined as in Formula (I) above.

In another embodiment, the GGA derivative provided and/or utilized is of Formula (I-D), (I-E), or (I-F):

[chemical structure]

wherein R⁶-R⁸ are defined as in Formula (I) above.

In another embodiment, the GGA derivative provided and/or utilized is of Formula (I-G), (I-H), or (I-I):

[chemical structure]

as a substantially pure trans isomer around the 2,3 double bond wherein R⁶-R⁸ are defined as in Formula (I) above.

In a preferred embodiment, R⁶ is C₆-C₁₀ aryl, such as naphthyl. In another preferred embodiment, R⁶ is a heteroaryl, such as quinolinyl.

In another aspect, the GGA derivative provided and/or utilized in this invention is of Formula (II):

[chemical structure]

or a pharmaceutically acceptable salt thereof, wherein
m is 0 or 1;
n is 0, or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently are hydrogen or C₁-C₆ alkyl;
Q₃ is —OH, —NR²²R²³—X—CO—NR²⁴R²⁵, —X—CS—NR²⁴R²⁵, or —X—SO₂—NR²⁴R²⁵;

### X is —O—, —NR²⁶—, or —CR²⁷R²⁸;

each R²² and R²³ independently is hydrogen; C₁-C₆ alkyl, optionally substituted with C₁-C₆ alkoxy;
and C₃-C₁₀ cycloalkyl;
each R²⁴ and R²⁵ independently is hydrogen, C₁-C₆ alkyl, optionally substituted with —CO₂H or an ester thereof, C₁-C₆ alkoxy, oxo, —OH, —CR═CR₂, —C≡CR, C₃-C₁₀ cycloalkyl, C₃-C₈ heterocyclyl, C₆-C₁₀ aryl, C₂-C₁₀ heteroaryl, wherein each R independently is hydrogen or C₁-C₆ alkyl;

C₃-C₁₀ cycloalkyl;

C₃-C₈ heterocyclyl;

C₆-C₁₀ aryl; or

C₂-C₁₀ heteroaryl;

wherein each cycloalkyl, heterocyclyl, aryl, or heteroaryl is optionally substituted with 1-3 alkyl groups; —CF₃, 1-3 halo, preferably, chloro or fluoro, groups; 1-3 nitro groups; 1-3 C₁-C₆ alkoxy groups; —CO-phenyl; or —NR¹⁸R¹⁹;
each R¹⁸ and R¹⁹ independently is hydrogen; C₁-C₆ alkyl, optionally substituted with —CO₂H or an ester thereof, C₁-C₆ alkoxy, oxo, —CR═CR₂, —CCR, C₃-C₁₀ preferably C₃-C₈ cycloalkyl, C₃-C₈ heterocyclyl, C₆-C₁₀ aryl, or C₂-C₁₀ heteroaryl, wherein each R independently is hydrogen or C₁-C₆ alkyl; C₃-C₁₀ cycloalkyl; C₃-C₈ heterocyclyl; C₆-C₁₀ aryl; or C₂-C₁₀ heteroaryl; wherein each cycloalkyl, heterocyclyl, aryl, or heteroaryl is optionally substituted with 1-3 alkyl groups, optionally substituted with 1-3 halo, preferably, fluoro, groups, where R¹⁸ and R¹⁹ together with the nitrogen atom they are attached to form a 5-7 membered heterocycle;
R²⁶ is hydrogen or together with R²⁴ or R²⁵ and the intervening atoms form a 5-7 membered heterocyclic ring optionally substituted with 1-3 C₁-C₆ alkyl groups; and
each R²⁷ and R²⁸ independently are hydrogen, C₁-C₆ alkyl, —COR⁸¹ or —CO₂R⁸¹, or R²⁷ together with R²⁴ or R²⁵ and the intervening atoms form a 5-7 membered heterocyclyl ring optionally substituted with 1-3 C₁-C₆ alkyl groups.

As used herein, the compound of Formula (II) includes optical isomers such as enantiomers and diastereomers. As also used herein, an ester refers preferably to a phenyl or a C₁-C₆ alkyl ester, which phenyl or alkyl group is optionally substituted with a amino group.

In one embodiment, Q₃ is —NR²²R²³—X—CO—NR²⁴R²⁵, —X—CS—NR²⁴R²⁵, or —X—SO₂—NR²⁴R²⁵. In another embodiment, Q₃ is —X—CO—NR²⁴R²⁵, —X—CS—NR²⁴R²⁵, or —X—SO₂—NR²⁴R²⁵. In another embodiment, Q₃ is —NR²²R²³. In another embodiment, Q₃ is —OH.

In one embodiment, the compound of Formula (II) is of formula:

[chemical structure]

wherein R¹, R², R³, R⁴, R⁵, and Q₃ are defined as in any aspect or embodiment herein.

In another embodiment, the GGA derivative provided and/or utilized is of formula:

[chemical structure]

wherein R¹, R², R⁴, R⁵, and Q₃ are defined as in any aspect and embodiment here.

In one embodiment, the compound of Formula (II) is of formula:

[chemical structure]

wherein R¹, R², R³, R⁴, R⁵, and Q₃ are defined as in any aspect or embodiment herein.

In another embodiment, the GGA derivative provided and/or utilized is of formula:

[chemical structure]

wherein R¹, R², R⁴, R⁵, m, n, X, R²⁴ and R²⁵ are defined as in any aspect and embodiment here.

In another embodiment, the GGA derivative provided and/or utilized is of formula:

[chemical structure]

wherein R¹, R², R⁴, R⁵, m, n, and R²⁴ are defined as in any aspect and embodiment here.

In another embodiment, the GGA derivative provided and/or utilized is of formula:

[chemical structure]

wherein R²⁴ is defined as in any aspect and embodiment here.

In another embodiment, the GGA derivative provided and/or utilized is of formula:

[chemical structure]

wherein R²⁴ is defined as in any aspect and embodiment here.

In another embodiment, the GGA derivative provided and/or utilized is of formula:

[chemical structure]

wherein R²⁴ is defined as in any aspect and embodiment here.

In another embodiment, the GGA derivative provided and/or utilized is of formula:

[chemical structure]

wherein R²⁴ is defined as in any aspect and embodiment here.

In another embodiment, the GGA derivative provided and/or utilized is of formula:

[chemical structure]

wherein R²⁴ and R²⁵ are defined as in any aspect and embodiment here.

In another embodiment, the GGA derivative provided and/or utilized is of formula:

[chemical structure]

wherein R²⁴ is defined as in any aspect and embodiment here.

In another embodiment, the GGA derivative provided and/or utilized is of formula:

[chemical structure]

wherein R²⁴ and R²⁵ are defined as in any aspect and embodiment here.

In one embodiment, m is 0. In another embodiment, m is 1.

In another embodiment, n is 0. In another embodiment, n is 1. In another embodiment, n is 2.

In another embodiment, m+n is 1. In another embodiment, m+n is 2. In another embodiment, m+n is 3.

In another embodiment, R¹ and R² are independently C₁-C₆ alkyl. In another embodiment, R¹ and R² independently are methyl, ethyl, or isopropyl.

In another embodiment, R¹ and R² together with the carbon atom they are attached to form a C₅-C₇ cycloalkyl ring optionally substituted with 1-3 C₁-C₆ alkyl groups. In another embodiment, R¹ and R² together with the carbon atom they are attached to form a ring that is:

[chemical structure]

In another embodiment, R³, R⁴, and R⁵ are independently C₁-C₆ alkyl. In another embodiment, one of R³, R⁴, and R⁵ are alkyl, and the rest are hydrogen: In another embodiment, two of R³, R⁴, and R⁵ are alkyl, and the rest are hydrogen. In another embodiment, R³, R⁴, and R⁵ are hydrogen. In another embodiment, R³, R⁴, and R⁵ are methyl.

In another embodiment, Q₃ is —X—CO—NR²⁴R²⁵. In another embodiment, Q₃ is —X—CS—NR²⁴R²⁵. In another embodiment, Q₃ is —X—SO₂—NR²⁴R²⁵. In another embodiment, Q₃ is —OCONHR²⁴, —OCONR²⁴R²⁵, —NHCONHR²⁴, —NHCONR²⁴R²⁵, —OCSNHR²⁴, —OCSNR²⁴R²⁵, —NHCSNHR²⁴, or —NHCSNR²⁴R²⁵.

In another embodiment, X is —O—. In another embodiment, X is —NR²⁶—. In another embodiment, X is or —CR²⁷R²⁸.

In another embodiment, one of R²⁴ and R²⁵ is hydrogen. In another embodiment, one or both of R²⁴ and R²⁵ are C₁-C₆ alkyl. In another embodiment, one or both of R²⁴ and R²⁵ are C₁-C₆ alkyl, optionally substituted with an R²⁰ group, wherein R²⁰ is —CO₂H or an ester thereof, C₁-C₆ alkyl, C₃-C₁₀ cycloalkyl, C₃-C₈ heterocyclyl, C₆-C₁₀ aryl, or C₂-C₁₀ heteroaryl. In another embodiment, one or both of R²⁴ and R²⁵ are C₃-C₁₀ cycloalkyl. In another embodiment, one or both of R²⁴ and R²⁵ are C₃-C₁₀ cycloalkyl substituted with 1-3 alkyl groups. In another embodiment, one or both of R²⁴ and R²⁵ are C₃-C₈ heterocyclyl. In another embodiment, one or both of R²⁴ and R²⁵ are C₆-C₁₀ aryl. In another embodiment, one or both of R²⁴ and R²⁵ are C₂-C₁₀ heteroaryl. In another embodiment, R²⁴ and R²⁵ together with the nitrogen atom they are attached to form a 5-7 membered heterocycle.

In another embodiment, R²⁰ is —CO₂H or an ester thereof. In another embodiment, R²⁰ is C₁-C₆ alkyl. In another embodiment, R²⁰ is C₃-C₁₀ cycloalkyl. In another embodiment, R²⁰ is C₃-C₈ heterocyclyl. In another embodiment, R²⁰ is C₆-C₁₀ aryl. In another embodiment, R²⁰ is or C₂-C₁₀ heteroaryl.

In another embodiment, the GGA derivative provided and/or utilized is of formula (II):

[chemical structure]

or a pharmaceutically acceptable salt thereof, wherein
   m is 0 or 1;
   n is 0, 1, or 2;
   each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
   each of R³, R⁴, and R⁵ independently are hydrogen or C₁-C₆ alkyl;
   Q₃ is —X—CO—NR²⁴R²⁵ or —X—SO₂—NR²⁴R²⁵;
   X is —O—, —NR²⁶—, or —CR²⁷R²⁸;
   R²⁶ is hydrogen or together with R²⁴ or R²⁵ and the intervening atoms form a 5-7 membered ring optionally substituted with 1-3 C₁-C₆ alkyl groups;
   each R²⁷ and R²⁸ independently are hydrogen, C₁-C₆ alkyl, —COR⁸¹ or —CO₂R⁸¹, or R²⁷ together with R²⁴ or R²⁵ and the intervening atoms form a 5-7 membered cycloalkyl or heterocyclyl ring optionally substituted with 1-3 C₁-C₆ alkyl groups;
   each R²⁴ and R²⁵ independently is
   hydrogen,
   C₁-C₆ alkyl, optionally substituted with —CO₂H or an ester thereof, C₃-C₁₀ preferably C₃-C₈ cycloalkyl, C₃-C₈ heterocyclyl, C₆-C₁₀ aryl, or C₂-C₁₀ heteroaryl,
   C₃-C₁₀ cycloalkyl,
   C₃-C₈ heterocyclyl,
   C₆-C₁₀ aryl, or
   C₂-C₁₀ heteroaryl,
wherein each cycloalkyl, heterocyclyl, aryl, or heteroaryl is optionally substituted with 1-3 C₁-C₆ alkyl groups, or R²⁴ and R²⁵ together with the nitrogen atom they are attached to form a 5-7 membered heterocycle.

In another embodiment, provided herein are compounds of formula:

[chemical structure]

In another aspect, the GGA derivative provided and/or utilized herein is of Formula III:

[chemical structure]

or a pharmaceutically acceptable salt of each thereof, wherein

m is 0 or 1;

n is 0, 1, or 2;

each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;

each of R³, R⁴, and R⁵ independently are hydrogen or C₁-C₆ alkyl;

Q₄ is selected from the group consisting of:

[chemical structure]

when X¹ is bonded via a single bond, X¹ is —O—, —NR³¹—, or —CR³²R³³—, and when X¹ is bonded via a double bond, X¹ is —CR³²—;

Y¹ is hydrogen, —OH or Y² is —OH, —OR¹¹ or —NHR¹², or Y¹ and Y² are joined to form an oxo group (═O), an imine group (═NR¹³), a oxime group (═N—OR¹⁴), or a substituted or unsubstituted vinylidene (═CR¹⁶R¹⁷);

R³⁰ is C₁-C₆ alkyl optionally substituted with 1-3 alkoxy or 1-5 halo group, C₂-C₆ alkenyl, C₂-C₆ alkynyl, C₃-C₁₀ cycloalkyl, C₆-C₁₀ aryl, C₃-C₈ heterocyclyl, or C₂-C₁₀ heteroaryl, wherein each cycloalkyl or heterocyclyl is optionally substituted with 1-3 C₁-C₆ alkyl groups, or wherein each aryl or heteroaryl is independently substituted with 1-3 C₁-C₆ alkyl or nitro groups, or R³⁰ is —NR³⁴R³⁵;

R³¹ is hydrogen or together with R³⁰ and the intervening atoms form a 5-7 membered ring optionally substituted with 1-3 C₁-C₆ alkyl groups;

each R³² and R³³ independently are hydrogen, C₁-C₆ alkyl, —COR⁸¹ or —CO₂R⁸¹, or R³² together with R³⁰ and the intervening atoms form a 5-7 membered cycloalkyl or heterocyclyl ring optionally substituted with oxo or 1-3 C₁-C₆ alkyl groups;

R¹⁰ is C₁-C₆ alkyl;

R¹¹ and R¹² are independently C₁-C₆ alkyl, C₃-C₁₀ cycloalkyl, —CO₂R¹⁵, or —CON(R¹⁵)₂, or R¹⁰ and R¹¹ together with the intervening carbon atom and oxygen atoms form a heterocycle optionally substituted with 1-3 C₁-C₆ alkyl groups;

R¹³ is C₁-C₆ alkyl or C₃-C₁₀ cycloalkyl optionally substituted with 1-3 C₁-C₆ alkyl groups;

R¹⁴ is hydrogen, C₃-C₈ heterocyclyl, or C₁-C₆ alkyl optionally substituted with a —CO₂H or an ester thereof or a C₆-C₁₀ aryl, C₂-C₆ alkenyl, C₂-C₆ alkynyl, C₃-C₁₀ cycloalkyl, or a C₃-C₈ heterocyclyl, wherein each cycloalkyl, heterocyclyl, or aryl, is optionally substituted with 1-3 alkyl groups;

each R¹⁵ independently are hydrogen, C₃-C₁₀ cycloalkyl, C₁-C₆ alkyl optionally substituted with 1-3 substituents selected from the group consisting of —CO₂H or an ester thereof, aryl, or C₃-C₈ heterocyclyl, or two R¹⁵ groups together with the nitrogen atom they are bonded to form a 5-7 membered heterocycle;

R¹⁶ is hydrogen or C₁-C₆ alkyl;

R¹⁷ is hydrogen, C₁-C₆ alkyl substituted with 1-3 hydroxy groups, —CHO, or is CO₂H or an ester thereof;

each R³⁴ and R³⁵ independently is hydrogen, C₁-C₆ alkyl, optionally substituted with —CO₂H or an ester thereof, C₃-C₁₀ cycloalkyl, C₃-C₈ heterocyclyl, C₆-C₁₀ aryl, or C₂-C₁₀ heteroaryl, or is C₃-C₁₀ cycloalkyl, C₃-C₈ heterocyclyl, C₆-C₁₀ aryl, or C₂-C₁₀ heteroaryl, wherein each cycloalkyl, heterocyclyl, aryl, or heteroaryl is optionally substituted with 1-3 alkyl groups, or R³⁴ and R³⁵ together with the nitrogen atom they are attached to form a 5-7 membered heterocycle; and

each R⁸¹ independently is C₁-C₆ alkyl.

In one embodiment, m is 0. In another embodiment, m is 1. In another embodiment, n is 0. In another embodiment, n is 1. In another embodiment, n is 2.

In one embodiment, the compound of Formula (III) is of formula:

[chemical structure]

wherein Q₄, R¹, R², R³, R⁴, R⁵, R³⁰, X¹, Y¹, and Y² are defined as in any aspect or embodiment herein.

In one embodiment, the GGA derivative provided and/or utilized is of formula:

[chemical structure]

wherein R¹, R², R³, R⁴, R⁵, R³⁰, X¹, Y¹, and Y² are defined as in any aspect and embodiment here.

In another embodiment, the GGA derivative provided and/or utilized is of formula:

[chemical structure]

wherein R¹, R², R³, R⁴, R⁵, R³⁰, and Y² are defined as in any aspect and embodiment herein.

In another embodiment, the GGA derivative provided and/or utilized is of formula:

[chemical structure]

wherein R¹, R², R³, R⁴, R⁵, R³⁰ and X¹ are defined as in any aspect and embodiment herein.

In another embodiment, the GGA derivative provided and/or utilized is of formula:

[chemical structure]

wherein R¹, R², R⁴, R⁵, and Q₄ are defined as in any aspect and embodiment herein.

In another embodiment, the GGA derivative provided and/or utilized is of formula:

[chemical structure]

wherein R¹, R², R⁴, R⁵, m, n, X¹, and R³⁰ are defined as in any aspect and embodiment here.

In another embodiment, the GGA derivative provided and/or utilized is of formula:

[chemical structure]

wherein R¹, R², R⁴, R⁵, m, n, and R³⁴ are defined as in any aspect and embodiment here.

In another embodiment, the GGA derivative provided and/or utilized is of formula:

[chemical structure]

wherein R¹, R², R⁴, R⁵, R³⁰, m, n, and R¹⁵ are defined as in any aspect and embodiment here.

In another embodiment, each R¹ and R² are C₁-C₆ alkyl. In another embodiment, each R¹ and R² are methyl, ethyl, or isopropyl. In another embodiment, R¹ and R² together with the carbon atom they are attached to form a 5-6 membered ring optionally substituted with 1-3 C₁-C₆ alkyl groups. In another embodiment, R¹ and R² together with the carbon atom they are attached to form a ring that is:

[chemical structure]

In another embodiment, R³, R⁴, and R⁵ are C₁-C₆ alkyl. In another embodiment, one of R³, R⁴, and R⁵ are alkyl, and the rest are hydrogen. In another embodiment, two of R³, R⁴, and R⁵ are alkyl, and the rest are hydrogen. In another embodiment, R³, R⁴, and R⁵ are hydrogen. In another embodiment, R³, R⁴, and R⁵ are methyl.

In another embodiment, X¹ is O. In another embodiment, X¹ is —NR³¹. In another embodiment, R³¹ is hydrogen. In another embodiment, R³¹ together with R³⁰ and the intervening atoms form a 5-7 membered ring optionally substituted with 1-3 C₁-C₆ alkyl groups. In another embodiment, X¹ is —CR³²R³³—. In another embodiment, X¹ is —CR³²—. In another embodiment, each R³² and R³³ independently are hydrogen, C₁-C₆ alkyl, or —CO₂R⁸¹. In another embodiment, R³² is hydrogen, and R³³ is hydrogen, C₁-C₆ alkyl, —COR⁸¹, or —CO₂R⁸¹.

In another embodiment, R³³ is hydrogen. In another embodiment, R³³ C₁-C₆ alkyl. In another embodiment, R³³ is methyl. In another embodiment, R³³ is —CO₂R⁸¹. In another embodiment, R³³ is —COR⁸¹.

In another embodiment, R³² together with R³⁰ and the intervening atoms form a 5-7 membered ring. In another embodiment, the moiety:

[chemical structure]

which is “Q₄,” has the structure:

[chemical structure]

wherein R³³ is hydrogen, C₁-C₆ alkyl, or —CO₂R⁸¹ and n is 1, 2, or 3. Within these embodiments, in certain embodiments, R³³ is hydrogen or C₁-C₆ alkyl. In one embodiment, R³³ is hydrogen. In another embodiment, R³³ is C₁-C₆ alkyl.

In another embodiment, R³⁰ is C₁-C₆ alkyl. In another embodiment, R³⁰ is methyl, ethyl, butyl, isopropyl, or tertiary butyl. In another embodiment, R³⁰ is C₁-C₆ alkyl substituted with 1-3 alkoxy or 1-5 halo group. In another embodiment, R³⁰ is alkyl substituted with an alkoxy group. In another embodiment, R³⁰ is alkyl substituted with 1-5, preferably, 1-3, halo, preferably fluoro, groups.

In another embodiment, R³⁰ is NR³⁴R³⁵. In a preferred embodiment, R³⁵ is H. In a preferred embodiment, R³⁴ is C₁-C₆ alkyl, optionally substituted with a group selected from the group consisting of —CO₂H or an ester thereof, C₃-C₁₀ cycloalkyl, C₃-C₈ heterocyclyl, C₆-C₁₀ aryl, or C₂-C₁₀ heteroaryl. In another preferred embodiment, R³⁴ is C₃-C₁₀ cycloalkyl, C₃-C₈ heterocyclyl, C₆-C₁₀ aryl, or C₂-C₁₀ heteroaryl. In a more preferred embodiment, R³⁴ is C₃-C₁₀ cycloalkyl.

In another embodiment, R³⁰ is C₂-C₆ alkenyl or C₂-C₆ alkynyl. In another embodiment, R³⁰ is C₃-C₁₀ cycloalkyl. In another embodiment, R³⁰ is C₃-C₁₀ cycloalkyl substituted with 1-3 C₁-C₆ alkyl groups. In another embodiment, R³⁰ is cyclopropyl, cyclobutyl, cyclopentyl, cyclohexyl, or adamentyl. In another embodiment, R³⁰ is C₆-C₁₀ aryl or C₂-C₁₀ heteroaryl. In another embodiment, R³⁰ is a 5-7 membered heteroaryl containing at least 1 oxygen atom. In another embodiment, R³⁰ is C₆-C₁₀ aryl, C₃-C₈ heterocyclyl, or C₂-C₁₀ heteroaryl, wherein each aryl, heterocyclyl, or heteroaryl is optionally substituted with 1-3 C₁-C₆ alkyl groups.

In another embodiment, Y² is —O—R¹¹. In another embodiment, Y¹ and Y² are joined to form ═NR¹³. In another embodiment, Y¹ and Y² are joined to form ═NOR¹⁴. In another embodiment, Y¹ and Y² are joined to form (═O). In another embodiment, Y¹ and Y² are joined to form ═CR¹⁶R¹⁷.

In another embodiment, Q₄ is —CR³³COR³⁰. In another embodiment, R³⁰ is C₁-C₆ alkyl optionally substituted with an alkoxy group. In another embodiment, R³⁰ is C₃-C₈ cycloalkyl. In another embodiment, R³³ is hydrogen. In another embodiment, R³³ is C₁-C₆ alkyl. In another embodiment, R³³ is CO₂R⁸¹. In another embodiment, R³³ is COR⁸¹.

In another embodiment, Q₄ is —CH₂—CH(O—CONHR¹⁵)—R³⁰. In another embodiment, R¹⁵ is C₃-C₈ cycloalkyl. In another embodiment, R¹⁵ is C₁-C₆ alkyl optionally substituted with 1-3 substituents selected from the group consisting of —CO₂H or an ester thereof, aryl, or C₃-C₈ heterocyclyl. In a preferred embodiment within these embodiments, R³⁰ is C₁-C₆ alkyl.

In another embodiment, Q₄ is —O—CO—NHR³⁴. withing these embodiment, in another embodiment, R³⁴ is C₁-C₆ alkyl, optionally substituted with —CO₂H or an ester thereof, C₃-C₈ cycloalkyl, C₃-C₈ heterocyclyl, C₂-C₁₀ aryl, or C₂-C₁₀ heteroaryl. In yet another embodiment, R³⁴ is C₃-C₈ cycloalkyl, C₃-C₈ heterocyclyl, C₂-C₁₀ aryl, or C₂-C₁₀ heteroaryl.

In another embodiment, R¹⁴ is hydrogen. In another embodiment, R¹⁴ is C₁-C₆ alkyl optionally substituted with a —CO₂H or an ester thereof or a C₆-C₁₀ aryl optionally substituted with 1-3 alkyl groups. In another embodiment, R¹⁴ is C₂-C₆ alkenyl. In another embodiment, R¹⁴ is C₂-C₆ alkynyl In another embodiment, R¹⁴ is C₃-C₆ cycloalkyl optionally substituted with 1-3 alkyl groups. In another embodiment, R¹⁴ is C₃-C₈ heterocyclyl optionally substituted with 1-3 alkyl groups.

In another embodiment, preferably, R¹⁶ is hydrogen. In another embodiment, R¹⁷ is CO₂H or an ester thereof. In another embodiment, R¹⁷ is C₁-C₆ alkyl substituted with 1-3 hydroxy groups. In another embodiment, R¹⁷ is C₁-C₃ alkyl substituted with 1 hydroxy group. In another embodiment, R¹⁷ is —CH₂OH.

In another embodiment, R¹⁰ and R¹¹ together with the intervening carbon atom and oxygen atoms form a heteroycle of formula:

[chemical structure]

wherein q is 0 or 1, p is 0, 1, 2, or 3, and R³⁶ is C₁-C₆ alkyl.

In another embodiment, q is 1. In another embodiment, q is 2. In another embodiment, p is 0. In another embodiment, p is 1. In another embodiment, p is 2. In another embodiment, p is 3.

In one aspect, the GGA derivative provided and/or utilized herein is of Formula (IV):

[chemical structure]

or a tautomer thereof, or a pharmaceutically acceptable salt of each thereof, wherein
m is 0 or 1;
n is 0, 1, or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently are hydrogen or C₁-C₆ alkyl, or R⁵ and Q₅ together with the intervening carbon atoms form a 6 membered aryl ring, or a 5-8 membered cycloalkenyl ring, or a 5-14 membered heteroaryl or heterocycle, wherein each aryl, cycloalkenyl, heteroaryl, or heterocycle, ring is optionally substituted with 1-2 substituents selected from the group consisting of halo, hydroxy, oxo, —N(R⁴⁰)₂, and C₁-C₆ alkyl group;

Q₅ is —C(═O)H, —CO₂H or —CH═CHCO₂H, or a C₁-C₆ alkyl ester or acyl halide thereof, wherein the ester is optionally substituted with —CO-phenyl; a 6-10 membered aryl or a 5-14 membered heteroaryl or heterocycle containing up to 6 ring heteroatoms, wherein the heteroatom is selected from the group consisting of O, N, S, and oxidized forms of N and S, and further wherein the aryl, heteroaryl, or heterocyclyl ring is optionally substituted with 1-3 substituents selected from the group consisting of:

hydroxy, oxo, —N(R⁴⁰)₂, C₁-C₆ alkoxy group, and C₁-C₆ alkyl group,

wherein the alkyl group is optionally substituted with 1-3 substituents selected from hydroxy, NH₂, C₆-C₁₀ aryl, —CO₂H or an ester or an amide thereof,

a 5-9 membered heteroaryl containing up to 3 ring heteroatoms, wherein the heteroaryl is optionally substituted with 1-3 hydroxy, —N(R⁴⁰)₂, and C₁-C₆ alkyl group,

benzyl, and phenyl optionally substituted with 1-3 substituents selected from the group consisting of C₁-C₆ alkyl, C₁-C₆ alkoxy, hydroxy, and halo groups; and

wherein each R⁴⁰ independently is hydrogen or C₁-C₆ alkyl.

As used herein, the compound of Formula (IV) includes tautomers and optical isomers such as enantiomers and diastereomers. As also used herein, an ester refers preferably to a phenyl or a C₁-C₆ alkyl ester, which phenyl or alkyl group is optionally substituted with a amino group. As used herein, an amide refers preferably to a moiety of formula —CON(R⁴⁰)₂, wherein R⁴⁰ is defined as above.

In some embodiment, Q₆ is selected from a group consisting of oxazole, oxadiazole, oxazoline, azalactone, imidazole, diazole, triazole, and thiazole, wherein each heteroaryl or heterocycle is optionally substituted as disclosed above.

In one embodiment, the GGA derivative provided and/or utilized is of formula IV-A:

[chemical structure]

In another embodiment, the GGA derivative provided and/or utilized is of formula IV-B:

[chemical structure]

wherein R¹, R², R⁴, R⁵, and Q₅ are defined as in any aspect and embodiment here.

[chemical structure]

In another embodiment, Q₅ is selected from the group consisting of:

wherein R¹¹ is C₁-C₆ alkyl, C₆-C₁₀ aryl, C₃-C₈ heteroaryl, C₃-C₈ heteroaryl, C₃-C₁₀ cycloalkyl, and the alkyl group is optionally substituted with 1-3 C₆-C₁₀ aryl, C₃-C₈ heteroaryl, C₃-C₈ heteroaryl, C₃-C₁₀ cycloalkyl groups, and the aryl, heteroaryl, heteroaryl, cycloalkyl groups are optionally substituted with 1-3 C₁-C₆ alkyl, C₁-C₆ alkoxy, halo, preferably chloro or fluoro, C₆-C₁₀ aryl, C₃-C₈ heteroaryl, C₃-C₈ heteroaryl, C₃-C₁₀ cycloalkyl group.

In another embodiment, Q₅ is phenyl, optionally substituted as described herein. In another embodiment, Q₅ is benzimidazole, benzindazole, and such other 5-6 fused 9-membered bicyclic heteroaryl or heterocycle. In another embodiment, Q₅ is quinoline, isoquinoline, and such other 6-6 fused 10 membered heteroaryl or heterocycle. In another embodiment, Q₅ is benzodiazepine or a derivative thereof, such as, a benzodiazepinone. Various benzodiazepine and derivatives thereof are well known to the skilled artisan.

In another embodiment, m is 0. In another embodiment, m is 1.

In another embodiment, n is 0. In another embodiment, n is 1. In another embodiment, n is 2.

In another embodiment, m+n is 1. In another embodiment, m+n is 2. In another embodiment, m+n is 3.

In another embodiment, R¹ and R² are independently C₁-C₆ alkyl. In another embodiment, R¹ and R² independently are methyl, ethyl, or isopropyl.

In another embodiment, R¹ and R² together with the carbon atom they are attached to form a C₅-C₇ cycloalkyl ring optionally substituted with 1-3 C₁-C₆ alkyl groups. In another embodiment, R¹ and R² together with the carbon atom they are attached to form a ring that is:

[chemical structure]

In another embodiment, R³, R⁴, and R⁵ are independently C₁-C₆ alkyl. In another embodiment, one of R³, R⁴, and R⁵ are alkyl, and the rest are hydrogen. In another embodiment, two of R³, R⁴, and R⁵ are alkyl, and the rest are hydrogen. In another embodiment, R³, R⁴, and R⁵ are hydrogen. In another embodiment, R³, R⁴, and R⁵ are methyl.

In another embodiment, this invention provides a compound selected from the group consisting of:

[chemical structure]

wherein R¹¹ is defined as above.

In another aspect, GGA derivatives provided and/or utilized herein are of formula (V):

[chemical structure]

or a pharmaceutically acceptable salt thereof, wherein
m is 0 or 1;
n is 0, 1, or 2;
each R¹ and R² independently are C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently is hydrogen or C₁-C₆ alkyl;
Q₆ is selected from the group consisting of:

[chemical structure]

when X² is bonded via a single bond, X² is —O—, —NR⁵²—, or —CR⁵³R⁵⁴—, and when X² is bonded via a double bond, X² is —CR⁵³—;
Y¹¹ is hydrogen, —OH or —OR⁵⁵;
Y²² is —OH, —OR⁵⁶, —NHR⁵⁷, or —O—CO—NR⁵⁸R⁵⁹, or Y¹¹ and Y²² are joined to form an oxo group (═O), an imine group (═NR⁶⁰), a oxime group (═N—OR⁶¹), or a substituted or unsubstituted vinylidene (═CR⁶³R⁶⁴);
R⁵¹ is C₁-C₆ alkyl, C₁-C₆ alkyl substituted with 1-3 alkoxy or 1-5 halo groups, C₂-C₆ alkenyl, C₂-C₆ alkynyl, C₃-C₁₀ cycloalkyl, C₃-C₈ heterocyclyl, C₆-C₁₀ aryl, C₂-C₁₀ heteroaryl, or —NR⁶⁵R⁶⁶, wherein each cycloalkyl or heterocyclyl is optionally substituted with 1-3 C₁-C₆ alkyl groups, and wherein each aryl or heteroaryl is optionally substituted independently with 1-3 nitro and C₁-C₆ alkyl groups;
R⁵² is hydrogen or together with R⁵¹ and the intervening atoms form a 5-7 membered ring optionally substituted with 1-3 C₁-C₆ alkyl groups;
each R⁵³ and R⁵⁴ independently are hydrogen, C₁-C₆ alkyl, —COR⁸¹, —CO₂R⁸¹, or —CONHR⁸², or R⁵³ together with R⁵¹ and the intervening atoms form a 5-7 membered cycloalkyl or heterocyclyl ring optionally substituted with 1-3 C₁-C₆ alkyl groups;
R⁵⁵ is C₁-C₆ alkyl;
each R⁵⁶ and R⁵⁷ independently are C₁-C₆ alkyl, C₃-C₁₀ cycloalkyl, —CO₂R⁶², or —CON(R⁶²)₂; or R⁵⁵ and R⁵⁶ together with the intervening carbon atom and oxygen atoms form a heterocycle optionally substituted with 1-3 C₁-C₆ alkyl groups;
R⁵⁸ is: C₃-C₁₀ cycloalkyl, C₁-C₆ alkyl optionally substituted with —OH, CO₂H or an ester thereof, or C₃-C₁₀ cycloalkyl,

[chemical structure]

R⁵⁹ is hydrogen or C₁-C₆ alkyl;
R⁶⁰ is C₁-C₆ alkyl or C₃-C₁₀ cycloalkyl optionally substituted with 1-3 C₁-C₆ alkyl groups, or is:

[chemical structure]

R⁶¹ is hydrogen, C₃-C₈ heterocyclyl, or C₁-C₆ alkyl optionally substituted with a —CO₂H or an ester thereof or a C₆-C₁₀ aryl, C₂-C₆ alkenyl, C₂-C₆ alkynyl, C₃-C₁₀ cycloalkyl, or a C₃-C₈ heterocyclyl, wherein each cycloalkyl, heterocyclyl, or aryl, is optionally substituted with 1-3 alkyl groups;
each R⁶² independently are hydrogen, C₃-C₁₀ cycloalkyl, C₁-C₆ alkyl optionally substituted with 1-3 substituents selected from the group consisting of —CO₂H or an ester thereof, aryl, C₃-C₈ heterocyclyl, or two R⁶² groups together with the nitrogen atom they are bonded to form a 5-7 membered heterocycle;
R⁶³ is hydrogen or C₁-C₆ alkyl;
R⁶⁴ is hydrogen, C₁-C₆ alkyl substituted with 1-3 hydroxy groups, —CHO, or is CO₂H or an ester thereof;
one or both of R⁶⁵ and R⁶⁶ independently are hydrogen, C₁-C₆ alkyl, optionally substituted with —CO₂H or an ester thereof, C₃-C₁₀ cycloalkyl, C₃-C₈ heterocyclyl, C₂-C₁₀ aryl, or C₂-C₁₀ heteroaryl, or is C₃-C₁₀ cycloalkyl, C₃-C₈ heterocyclyl, C₆-C₁₀ aryl, or C₂-C₁₀ heteroaryl, wherein each cycloalkyl, heterocyclyl, aryl, or heteroaryl is optionally substituted with 1-3 alkyl groups, or R⁶⁵ and R⁶⁶ gether with the nitrogen atom they are bonded to form a 5-7 membered heterocycle, and if only one of R⁶⁵ and R⁶⁶ are defined as above, then the other one is

[chemical structure]

and
R⁸¹ is C₁-C₆ alkyl; and
R⁸² is

[chemical structure]

provided that, when X² is bonded via a single bond, and R⁵³ or R⁵⁴ is not —CONHR⁸², T¹¹ and Y²² are joined to form an imine group (═NR⁶⁰), and R⁶⁰ is:

[chemical structure]

or Y²² is —O—CO—NR⁵⁸R⁵⁹;
   or provided that, when Q₆ is:

[chemical structure]

and R⁵³ is not —CONHR⁸², Y²² is —O—CO—NR⁵⁸R⁵⁹;
   or provided that, when Q₆ is —O—CO—NR⁶⁵R⁶⁶, then at least one of R⁶⁵ and R⁶⁶ is:

[chemical structure]

In one embodiment, the GGA derivative provided and/or utilized are of formula:

[chemical structure]

In another aspect, the GGA derivatives useful according to this invention is selected from:

[chemical structure]

In one embodiment, the compounds provided herein excludes the compound of formula:

[chemical structure]

wherein L is 0, 1, 2, or 3, and R¹⁷ is CO₂H or an ester thereof, or is —CH₂OH, or is a C₁-C₆ alkyl ester of —CH₂OH.

In another embodiment, examples of compounds provided and/or utilized by this invention include certain compounds tabulated below. Compound ID numbers in Table 1 refer to synthetic schemes in Example 7.

TABLE 1

| Compound ID | |
|---|---|
| (see Example | |
| 7) | Structure |
| 1 | [chemical structure] |
| 2a | [chemical structure] |
| 2b | [chemical structure] |
| 2c | [chemical structure] |
| 2d | [chemical structure] |
| 2e | [chemical structure] |
| 2f | [chemical structure] |
| 2g | [chemical structure] |
| 2h | [chemical structure] |
| 2i | [chemical structure] |
| 2j | [chemical structure] |
| 2k | [chemical structure] |
| 2l | [chemical structure] |
| 4a | [chemical structure] |
| 4b | [chemical structure] |
| 4c | [chemical structure] |
| 6a | [chemical structure] |
| 6b | [chemical structure] |
| 7a | [chemical structure] |
| 7b | [chemical structure] |
| 7c | [chemical structure] |
| 7d | [chemical structure] |
| 7e | [chemical structure] |
| 7f | [chemical structure] |
| 7g | [chemical structure] |
| 7h | [chemical structure] |
| 7i | [chemical structure] |
| 7j | [chemical structure] |
| 7k | [chemical structure] |
| 7l | [chemical structure] |
| 7m | [chemical structure] |
| 7n | [chemical structure] |
| 7o | [chemical structure] |
| 7p | [chemical structure] |
| 7q | [chemical structure] |
| 7r | [chemical structure] |
| 7s | [chemical structure] |
| 7t | [chemical structure] |
| 7u | [chemical structure] |
| 7v | [chemical structure] |
| 7w | [chemical structure] |
| 7x | [chemical structure] |
| 7y | [chemical structure] |
| 7z | [chemical structure] |
| 7aa | [chemical structure] |
| 8a | [chemical structure] |
| 8b | [chemical structure] |
| 8c | [chemical structure] |
| 8d | [chemical structure] |
| 8e | [chemical structure] |
| 8f | [chemical structure] |
| 8g | [chemical structure] |
| 8h | [chemical structure] |
| 8i | [chemical structure] |
| 8j | [chemical structure] |
| 8k | [chemical structure] |
| 8l | [chemical structure] |
| 8m | [chemical structure] |
| 8n | [chemical structure] |
| 8o | [chemical structure] |
| 9a | [chemical structure] |
| 9b | [chemical structure] |
| 9c | [chemical structure] |
| 9d | [chemical structure] |
| 9e | [chemical structure] |
| 9f | [chemical structure] |
| 9g | [chemical structure] |
| 9h | [chemical structure] |
| 9i | [chemical structure] |
| 9j | [chemical structure] |
| 9k | [chemical structure] |
| 10a | [chemical structure] |
| 10b | [chemical structure] |
| 10c | [chemical structure] |
| 10d | [chemical structure] |
| 10e | [chemical structure] |
| 10f | [chemical structure] |
| 10g | [chemical structure] |
| 10h | [chemical structure] |
| 10i | [chemical structure] |
| 10j | [chemical structure] |
| 10k | [chemical structure] |
| 10l | [chemical structure] |
| 10m | [chemical structure] |
| 12 | [chemical structure] |
| 14 | [chemical structure] |
| 15 | [chemical structure] |
| 16 | [chemical structure] |
| 17a | [chemical structure] |
| 17b | [chemical structure] |
| 17c | [chemical structure] |
| 17d | [chemical structure] |
| 17e | [chemical structure] |
| 19 | [chemical structure] |
| 20a | [chemical structure] |
| 20b | [chemical structure] |
| 20c | [chemical structure] |
| 20d | [chemical structure] |
| 20e | [chemical structure] |
| 20f | [chemical structure] |
| 20g | [chemical structure] |
| 20h | [chemical structure] |
| 20i | [chemical structure] |
| 20j | [chemical structure] |
| 22 | [chemical structure] |
| 23a | [chemical structure] |
| 23b | [chemical structure] |
| 23c | [chemical structure] |
| 23d | [chemical structure] |
| 23e | [chemical structure] |
| 23f | [chemical structure] |
| 23g | [chemical structure] |
| 24 | [chemical structure] |
| 25 | [chemical structure] |
| 27a | [chemical structure] |
| 27b | [chemical structure] |
| 27c | [chemical structure] |
| 27d | [chemical structure] |
| 27e | [chemical structure] |
| 27f | [chemical structure] |
| 27g | [chemical structure] |
| 29a | [chemical structure] |
| 29b | [chemical structure] |
| 29c | [chemical structure] |
| 29d | [chemical structure] |
| 29e | [chemical structure] |
| 29f | [chemical structure] |
| 31 | [chemical structure] |
| 32 | [chemical structure] |
| 35a | [chemical structure] |
| 35b | [chemical structure] |
| 35c | [chemical structure] |
| 35d | [chemical structure] |
| 37a | [chemical structure] |
| 37b | [chemical structure] |
| 37c | [chemical structure] |
| 37d | [chemical structure] |
| 38a | [chemical structure] |
| 38b | [chemical structure] |
| 39 | [chemical structure] |
| 40a | [chemical structure] |
| 40b | [chemical structure] |
| 41 | [chemical structure] |
| 42 | [chemical structure] |
| 43 | [chemical structure] |

In another embodiment, examples of compounds provided and/or utilized by this invention include certain compounds tabulated below.

TABLE 2

| Compound | |
|---|---|
| ID | Chemical Structure |
| 51 | [chemical structure] |
| 52 | [chemical structure] |
| 54 | [chemical structure] |
| 55 | [chemical structure] |
| 56 | [chemical structure] |
| 57 | [chemical structure] |
| 58 | [chemical structure] |
| 59 | [chemical structure] |
| 60 | [chemical structure] |
| 61 | [chemical structure] |
| 62 | [chemical structure] |
| 63 | [chemical structure] |
| 64 | [chemical structure] |
| 65 | [chemical structure] |
| 66 | [chemical structure] |
| 67 | [chemical structure] |
| 68 | [chemical structure] |
| 69 | [chemical structure] |
| 70 | [chemical structure] |
| 71 | [chemical structure] |
| 72 | [chemical structure] |
| 73 | [chemical structure] |
| 74 | [chemical structure] |
| 75 | [chemical structure] |
| 76 | [chemical structure] |
| 77 | [chemical structure] |
| 78 | [chemical structure] |
| 6979 | [chemical structure] |
| 80 | [chemical structure] |
| 81 | [chemical structure] |
| 82 | [chemical structure] |
| 83 | [chemical structure] |
| 84 | [chemical structure] |
| 85 | [chemical structure] |
| 86 | [chemical structure] |
| 87 | [chemical structure] |
| 88 | [chemical structure] |
| 89 | [chemical structure] |
| 90 | [chemical structure] |
| 91 | [chemical structure] |
| 92 | [chemical structure] |
| 93 | [chemical structure] |
| 94 | [chemical structure] |
| 95 | [chemical structure] |
| 96 | [chemical structure] |
| 97 | [chemical structure] |
| 98 | [chemical structure] |
| 99 | [chemical structure] |
| 100 | [chemical structure] |
| 101 | [chemical structure] |
| 102 | [chemical structure] |
| 103 | [chemical structure] |
| 104 | [chemical structure] |
| 105 | [chemical structure] |
| 106 | [chemical structure] |
| 107 | [chemical structure] |
| 108 | [chemical structure] |
| 109 | [chemical structure] |
| 110 | [chemical structure] |
| 111 | [chemical structure] |
| 112 | [chemical structure] |
| 113 | [chemical structure] |
| 114 | [chemical structure] |
| 115 | [chemical structure] |
| 116 | [chemical structure] |
| 117 | [chemical structure] |
| 118 | [chemical structure] |
| 119 | [chemical structure] |
| 120 | [chemical structure] |
| 121 | [chemical structure] |
| 122 | [chemical structure] |
| 123 | [chemical structure] |
| 124 | [chemical structure] |
| 125 | [chemical structure] |
| 126 | [chemical structure] |
| 127 | [chemical structure] |
| 128 | [chemical structure] |
| 129 | [chemical structure] |
| 130 | [chemical structure] |
| 131 | [chemical structure] |
| 132 | [chemical structure] |
| 133 | [chemical structure] |
| 134 | [chemical structure] |
| 135 | [chemical structure] |
| 136 | [chemical structure] |
| 137 | [chemical structure] |
| 138 | [chemical structure] |
| 139 | [chemical structure] |
| 140 | [chemical structure] |
| 141 | [chemical structure] |
| 142 | [chemical structure] |
| 143 | [chemical structure] |
| 144 | [chemical structure] |
| 145 | [chemical structure] |
| 146 | [chemical structure] |
| 147 | [chemical structure] |

### Synthesis of GGA Derivatives

Certain methods for making GGA or certain GGA derivatives provided and/or utilized herein are described in PCT publication no. WO 2012/031028 and PCT application no. PCT/US2012/027147, each of which are incorporated herein by reference in its entirety. Other GGA derivatives can be prepared by appropriate substitution of reagents and starting materials, as will be well known to the skilled artisan upon reading this disclosure.

The reactions are preferably carried out in a suitable inert solvent that will be apparent to the skilled artisan upon reading this disclosure, for a sufficient period of time to ensure substantial completion of the reaction as observed by thin layer chromatography, ¹H-NMR, etc. If needed to speed up the reaction, the reaction mixture can be heated, as is well known to the skilled artisan. The final and the intermediate compounds are purified, if necessary, by various art known methods such as crystallization, precipitation, column chromatography, and the likes, as will be apparent to the skilled artisan upon reading this disclosure.

The compounds provided and/or utilized in this invention are synthesized, e.g., from a compound of formula (III-A):

[chemical structure]

wherein n, R¹-R⁵ and [symbol] are defined as in Formula (I) above, following various well known methods upon substitution of reactants and/or altering reaction conditions as will be apparent to the skilled artisan upon reading this disclosure. The compound of Formula (III-A) is itself prepared by methods well known to a skilled artisan, for example, and without limitation, those described in PCT Pat. App. Pub. No. WO 2012/031028 and PCT Pat. App. No. PCT/US2012/027147 (each supra). An illustrative and non-limiting method for synthesizing a compound of Formula (III-A), where n is 1, is schematically shown below.

[chemical structure]

Starting compound (iii), which is synthesized from compound (i) by adding isoprene derivatives as described here, is alkylated with a beta keto ester (iv), in the presence of a base such as an alkoxide, to provide the corresponding beta-ketoester (v). Compound (v) upon alkaline hydrolysis followed by decarboxylation provides ketone (vi). Keto compound (vi) is converted, following a Wittig Horner reaction with compound (vii), to the conjugated ester (viii). Compound (viii) is reduced, for example with LiAlH₄, to provide alcohol (ix).

As will be apparent to the skilled artisan, a compound of Formula (III), where n is 2, is synthesized by repeating the reaction sequence of alkylation with a beta-keto ester, hydrolysis, decarboxylation, Wittig-Horner olefination, and LiAlH₄ reduction.

Certain illustrative and non-limiting synthesis of compounds provided and/or utilized in this invention are schematically shown below. Compounds where Q¹ is —(C═S)— or —SO₂— are synthesized by substituting the carbonyl group of the reactants employed, as will be apparent to the skilled artisan.

R⁶ in the schemes below may also correspond to R³⁰ and R⁵¹ as defined herein. R⁷ in the schemes below may also correspond to R²⁶, R³¹ and R⁵² as defined herein. R⁸ in the schemes below may also correspond to R²⁷, R³² and R⁵³ as defined herein. R⁹ in the schemes below may also correspond to R²⁸, R³³ and R⁵⁴ as defined herein. R¹³ in the schemes below may also correspond to R⁵⁸ as defined herein. R¹⁴ in the schemes below may also correspond to R⁵⁹ as defined herein. R¹⁶ in the schemes below may also correspond to R⁶⁰ as defined herein. R¹⁸ in the schemes below may also correspond to R²⁴, R³⁴ and R⁶³ as defined herein. R¹⁹ in the schemes below may also correspond to R²⁵, R³⁵ and R⁶⁴ as defined herein. L is a leaving group as known to one of ordinary skill in the art.

[chemical structure]

As shown above, R^{E} is alkyl.

Compound (ix) with alcohol functionality is an intermediate useful for preparing the compounds provided and/or utilized in this invention. Compound (x), where L is an R⁵SO₂— group is made by reacting compound (ix) with R⁵SO₂Cl in the presence of a base. The transformation of compound (iii) to compound (x) illustrates methods of adding isoprene derivatives to a compound, which methods are suitable to make compound (iii) from compound (i). Intermediate (ix) containing various R¹-R⁵ substituents are prepared according to this scheme as exemplified herein below. The transformation of compound (iii) to compound (x) illustrates methods of adding isoprene derivatives to a compound, which methods are suitable to make compound (iii) from compound (i).

The intermediates prepared above are converted to the compounds provided and/or utilized in this invention as schematically illustrated below:

[chemical structure]

As used herein, for example, and without limitation, m is 0 or 1 and R¹-R⁵ are as defined herein, and are preferably alkyl, or more preferably methyl. Intermediate (ixa), prepared according to the scheme herein above, is converted to amino intermediate (ixb) via the corresponding bromide. Intermediates (ixa) and (ixb) are converted to the compounds provided and/or utilized in this invention by reacting with suitable isocyanates or carbamoyl chlorides, which are prepared by art known methods. The thiocarbamates and thioureas of this invention are prepared according to the methods described above and replacing the isocyanates or the carbamoyl chlorides with isothiocyanates (R¹⁸—N═C═S) or thiocarbamoyl chlorides (R¹⁸—NH—C(═S)Cl or R¹⁸R¹⁹N—C(═S)Cl). These and other compounds provided and/or utilized in this invention are also prepared by art known methods, which may require optional modifications as will be apparent to the skilled artisan upon reading this disclosure. Intermediates for synthesizing compounds provided and/or utilized in this invention containing various R¹-R⁵ substituents are illustrated in the examples section and/or are well known to the skilled artisan.

Certain GGA derivatives provided and/or utilized herein are synthesized as schematically shown below.

[chemical structure]

Certain compounds provided and/or utilized herein are obtained by reacting compound (x) with the anion Q(−), which can be generated by reacting the compound QH with a base. Suitable nonlimiting examples of bases include hydroxide, hydride, amides, alkoxides, and the like. Various compounds provided and/or utilized in this invention, wherein the carbonyl group is converted to an imine, a hydrazone, an alkoxyimine, an enolcarbamate, a ketal, and the like, are prepared following well known methods.

Other methods for making the compounds provided and/or utilized in this invention are schematically illustrated below:

[chemical structure]

The metallation is performed, by reacting the ketone with a base such as dimsyl anion, a hindered amide base such as diisopropylamide, or hexamethyldisilazide, along with the corresponding metal cation, M. The amino carbonyl chloride or the isocyanate is prepared, for example, by reacting the amine (R¹⁴)₂NH with phosgene or an equivalent reagent well known to the skilled artisan.

[chemical structure]

The beta keto ester is hydrolyzed while ensuring that the reaction conditions do not lead to decarboxylation. The acid is activated with various acid activating agent well known to the skilled artisan such as carbonyl diimodazole, or O-benzotriazole-N,N,N′,N′-tetramethyl-uronium-hexafluoro-phosphate (HBTU) and reacted with the amine.

[chemical structure]

Various other compounds provided and/or utilized in this invention are prepared from the compounds made in the scheme above based on art known methods.

[chemical structure]

As shown above, R^{E} is alkyl.

The intermediates prepared above are converted to the compounds provided and/or utilized in this invention as schematically illustrated below:

[chemical structure]

Compound (viii) is hydrolyzed to the carboxylic acid (x), which is then converted to the acid chloride (xi). Compound (xi) is reacted with a suitable nucleophile such as a hydrazide, a hydroxylamine, an amino alcohol, or an amino acid, and the intermediate dehydrated to provide a compound of Formula (IV). Alternatively, the allylic alcohol (ix) is oxidized to the aldehyde (xi), which is then reacted with a cyanohydrin or cyanotosylmethane to provide further compounds provided and/or utilized in this invention.

GGA derivatives provided and/or utilized in this invention can also be synthesized employing art known methods and those disclosed here by alkene-aryl, alkene-heteroaryl, or alkene-akene couplings such as Heck, Stille, or Suzuki coupling. Such methods can use (vi) to prepare intermediate (xii) that can undergo Heck, Stifle, or Suzuki coupling under conditions well known to the skilled artisan to provide compounds provided and/or utilized in this invention.

[chemical structure]

Higher and lower isoprenyl homologs of intermediates (x), (xi), and (xii), which are prepared following the methods disclosed here, can be similarly employed to prepare other compounds provided and/or utilized in this invention.

Compounds provided and/or utilized in this invention are also prepared as shown below

[chemical structure]

L is a leaving group and Q₅ are as defined herein, Ar is a preferably an aryl group such as phenyl, the base employed is an alkoxide such as tertiarybutoxide, a hydride, or an alkyl lithium such as n-butyl lithium. Methods of carrying out the steps shown above are well known to the skilled artisan, as are conditions, reagents, solvents, and/or additives useful for performing the reactions and obtaining the compound of Formula (IV) in the desired stereochemistry.

Other methods for making the compounds provided and/or utilized in this invention are schematically illustrated below:

[chemical structure]

The metallation is performed, by reacting the ketone with a base such as dimsyl anion, a hindered amide base such as diisopropylamide, or hexamethyldisilazide, along with the corresponding metal cation, M. The amino carbonyl chloride or the isocyanate is prepared, for example, by reacting the amine R¹³R¹⁴NH with phosgene or an equivalent reagent well known to the skilled artisan.

[chemical structure]

The beta keto ester is hydrolyzed while ensuring that the reaction conditions do not lead to decarboxylation. The acid is activated with various acid activating agent well known to the skilled artisan such as carbonyl diimodazole, or O-benzotriazole-N,N,N′,N′-tetramethyl-uronium-hexafluoro-phosphate (HBTU) and reacted with the amine. Certain other methods of preparing the conjugates are shown below.

[chemical structure]

As shown above, R is a memantine or a riluzole residue.
Polyprenyl amine—GGA derivatives can be prepared by reductive amination employing the appropriate polyprenyl aldehyde, a primary or secondary amine and a borohydride reducing agent, as is well known to the skilled artisan. The reaction can be carried out in THF or diethyl ether, optionally in presence of a protic acid, preferably a mild protic acid catalyst.

### Pharmaceutical Compositions

In another aspect, this invention provides a composition comprising a GGA or a GGA derivative provided herein and a pharmaceutically acceptable excipient.

Such compositions can be formulated for different routes of administration. Although compositions suitable for oral delivery will probably be used most frequently, other routes that may be used include transdermal, intravenous, intraarterial, pulmonary, rectal, nasal, vaginal, lingual, intramuscular, intraperitoneal, intracutaneous, intracranial, and subcutaneous routes. Suitable dosage forms for administering the GGA or GGA derivatives of this invention include tablets, capsules, pills, powders, aerosols, suppositories, parenterals, and oral liquids, including suspensions, solutions and emulsions. Sustained release dosage forms may also be used, for example, in a transdermal patch form. All dosage forms may be prepared using methods that are standard in the art (see e.g., Remington's Pharmaceutical Sciences, 16^{th} ed., A. Oslo editor, Easton Pa. 1980).

Pharmaceutically acceptable excipients are non-toxic, aid administration, and do not adversely affect the therapeutic benefit of the compound of this invention. Such excipients may be any solid, liquid, semi-solid or, in the case of an aerosol composition, gaseous excipient that is generally available to one of skill in the art. Pharmaceutical compositions in accordance with the invention are prepared by conventional means using methods known in the art.

The compositions disclosed herein may be used in conjunction with any of the vehicles and excipients commonly employed in pharmaceutical preparations, e.g., talc, gum arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerin and the like.

Solid pharmaceutical excipients include starch, cellulose, hydroxypropyl cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. In certain embodiments, the compositions provided herein comprises one or more of α-tocopherol, gum arabic, and/or hydroxypropyl cellulose.

In one embodiment, this invention provides sustained release formulations such as drug depots or patches comprising an effective amount of a compound provided herein. In another embodiment, the patch further comprises gum Arabic or hydroxypropyl cellulose separately or in combination, in the presence of alpha-tocopherol. Preferably, the hydroxypropyl cellulose has an average MW of from 10,000 to 100,000. In a more preferred embodiment, the hydroxypropyl cellulose has an average MW of from 5,000 to 50,000.

In one embodiment, this invention provides pharmaceutical compositions in the form of an enterocoated capsule or tablet that facilitates increased delivery of GGA to the intestine.

Compounds and pharmaceutical compositions of this invention maybe used alone or in combination with other compounds. When administered with another agent, the co-administration can be in any manner in which the pharmacological effects of both are manifest in the patient at the same time. Thus, co-administration does not require that a single pharmaceutical composition, the same dosage form, or even the same route of administration be used for administration of both the compound of this invention and the other agent or that the two agents be administered at precisely the same time. However, co-administration will be accomplished most conveniently by the same dosage form and the same route of administration, at substantially the same time. Obviously, such administration most advantageously proceeds by delivering both active ingredients simultaneously in a novel pharmaceutical composition in accordance with the present invention.

In some embodiments, a compound of this invention can be used as an adjunct to conventional drug therapy of the conditions described herein.

### EXAMPLES

The following examples of serve to illustrate the invention without limiting its scope.

### Example 1

### Time Course of CNS-102 Induced HSP70 Expression In Vivo

The time course of protein expression, as measured by western blot for HSP70, was determined in triplicate for hippocampus, and cortex tissue samples taken from each of 5 animals per group at each of four time points (24, 48, 72, and 96 h) after treatment with either PBS or 12 mg/kg CNS-102, administered orally. The average expression for each treatment group is calculated at each time point for each tissue using PROC MIXED in SAS and are tabulated, along with the difference (delta) between treatment averages and a p-value comparing the difference to zero, below.

HSP70 Expression Following Administration of CNS-102 vs PBS

| | Treatment; | | |
|---|---|---|---|
| CNS-PBS | Statistic | Cortex | Hippocampus |
| 24 hours | CNS | −0.016 | −0.159 |
| | PBS | −0.256 | −0.072 |
| | delta | 0.24 | −0.088 |
| | p-value | 0.002 | 0.16 |
| 48 hours | CNS | −0.45 | 0.02 |
| | PBS | −0.56 | −0.18 |
| | delta | 0.11 | 0.2 |
| | p-value | 0.15 | 0.14 |
| 72 hours | CNS | 0.14 | −0.06 |
| | PBS | 0.01 | −0.2 |
| | delta | 0.13 | 0.14 |
| | p-value | 0.13 | 0.032 |
| 96 hours | CNS | 0.04 | −0.32 |
| | PBS | −0.15 | −0.49 |
| | delta | 0.19 | 0.17 |
| | p-value | 0.09 | 0.07 |

Expression of HSP70 was observed after CNS-102 administration and the difference between CNS-102 and PBS induced expression (delta, in the table) in both the cortex at 24 h and the hippocampus at 72 h was statistically significant (bolded in the table).

These results demonstrate that CNS-102 induces expression of HSP70 as measured in the cortex 24 h after administration while in the hippocampus the level of HSP70 was not significant until 72 h after administration. No significant levels of HSP70 were found in the cortex after 24 h, however since no time points before 24 h were taken, it may be that HSP70 is expressed earlier. In the hippocampus the expression appears to peak after 48 h with significant levels measured at 72 hours.

CNS-102 at 12 mg/kg or PBS was administered orally to Sprague-Dawley rats and the time course of HSP70 protein expression in tissues, was measured by ELISA. HSP70 protein expression was determined for lung, testicle, spleen, liver, kidney, blood plasma, skin, peripheral blood monocytes, heart, eye, muscle, intestine, and stomach at each of three time points (8 h, 17 h, 24 h).

TABLE
Time Course of HSP70 expression as measured by ELISA in select
tissues following 12 mg/kg p.o of CNS-102

HSP70 Fold Induction
vs. Vehicle Control

| | 8 h | 17 h | 24 h | 48 h |
|---|---|---|---|---|
| testicle | 1.10 | 1.03 | 0.94 | |
| spleen | 0.64 | 1.09 | 1.04 | |
| liver | 1.24 | 1.00 | 0.89 | |
| kidney | 1.11 | 1.08 | 0.92 | |
| plasma | 0.88 | 1.09 | 1.05 | |
| PBMC | 1.67 | 1.05 | 1.09 | |
| heart | | 1.89 | 0.60 | |
| Intestine | 1.63 | 1.26 | 0.64 | |
| Stomach | 1.07 | 1.30 | 0.96 | |

### Example 2

### Treatment of Inflammatory Bowel Disease (IBD) with GGA or a GGA Derivative

A pharmaceutical composition comprising GGA or a GGA derivative as described herein is prepared. A subject is diagnosed with mild to moderate IBD. The subject receives a daily administration of GGA or a GGA derivative, or a pharmaceutically acceptable salt thereof. Subjects are treated for 12 weeks. Subjects keep daily diaries and record the number and nature of bowel movements. The effect of the treatments is assessed by grading clinical symptoms of fecal blood, mucus, and urgency. In addition, sigmoidoscopic assessment and biopsies are performed, and efficacy of treatment assessed, based on grading of sigmoidoscopic and degree of histological inflammation in rectal biopsy specimens. Safety is assessed based on spontaneous side effect reporting.

It is contemplated that GGA or a GGA derivative, or a pharmaceutically acceptable salt thereof, of this example will demonstrate efficacy in inflammatory bowel disease IBD in terms of both treating the condition and maintaining remission from disease symptoms.

### Example 3

### Treatment of Inflammatory Bowel Disease (IBD) with GGA or a GGA Derivative in Gastrectomized Patients

A pharmaceutical composition comprising GGA or a GGA derivative as described herein is prepared. A subject is diagnosed with mild to moderate IBD following gastrectomy. The subject receives a daily administration of GGA or a GGA derivative, or a pharmaceutically acceptable salt thereof. Subjects are treated for 12 weeks. Subjects keep daily diaries and record the number and nature of bowel movements. The effect of the treatments is assessed by grading clinical symptoms of fecal blood, mucus, and urgency. In addition, sigmoidoscopic assessment and biopsies are performed, and efficacy of treatment assessed, based on grading of sigmoidoscopic and degree of histological inflammation in rectal biopsy specimens. Safety is assessed based on spontaneous side effect reporting.

It is contemplated that GGA or a GGA derivative, or a pharmaceutically acceptable salt thereof, of this example will demonstrate efficacy in inflammatory bowel disease IBD in terms of both treating the condition and maintaining remission from disease symptoms.

### Example 4

### GGA and Derivatives Thereof Protect Intestinal Epithelial Cells from Oxidative Stress In Vitro

Rat intestinal epithelial cell line (IEC-18) cells are pretreated with GGA or a GGA derivative and then subjected to injury induced by NH₂Cl. Cell viability is assessed, and endogenous HSP70 levels are determined by enzyme-linked immunosorbent assay in IEC-18 cells. Treatment with GGA or a derivative thereof rapidly elevates HSP70 levels and protects against NH₂Cl-induced injury in IEC-18 cells.

### Example 5

### GGA and GGA Derivatives Protect Mice from Dextran Sulfate Sodium (DSS)-Induced Colitis

BALB/c mice are given 3% DSS solution orally for 7 days to induce colitis. The disease activity of colitis is assessed clinically every day, and histology in the colon is evaluated at 7 days post-DSS. The levels of myeloperoxidase (MPO) activity, tumor necrosis factor (TNF)-alpha and interferon (IFN)-gamma in the colon tissues are also examined. In addition, expression of HSPs 25, 40, 70 and 90 in the colon tissue is determined by Western blot analysis or ELISA. GGA or a GGA derivative is administered orally to mice when treatment of DSS is initiated. GGA or a derivateive thereof significantly reduces the clinical severity of colitis and suppresses the levels of MPO activity, TNF-alpha and IFN-gamma induced by DSS in the colon. On the other hand, GGA enhances the expression of HSP70 in the colon of mice given DSS.

### Example 6

### Prevention of Acute Liver Damage after Hepatectomy

Acute liver failure after massive hepatectomy remains a challenging problem. Male Wister rats weighing 230-260 g are obtained. After an overnight fast, GGA or a GGA derivative (as an emulsion with 5% gum arabic and 0.004% α-tocophenol) or vehicle (5% gum arabic emulsion with 0.004% α-tocophenol) is intragastrically administrated into rats 4 h prior to the operation. After rats are anesthetized, 90% hepatectomy is performed. Briefly, the left, median, right-upper, and right-lower lobes are removed, leaving the caudate lobes, which represent 10-11% of the original liver mass. Liver specimens and blood samples are collected after laparotomy and exsanguinations under deep anesthesia immediately before (0) and 4,8, 12, and 24 h after the operation. Small pieces of liver tissue are immediately stored in an RNeasy stabilization kit (Qiagen, Hilden, Germany). Sera are immediately separated, and the activities of alanine (ALT) and aspartate (AST) aminotransferases are measured.

A single oral administration of GGA or a GGA derivative significantly suppresses the release of aminotransferases and improves survival compared with vehicle administration. Gene expression and immunoblot analyses shows that, in addition to HSP70 and HSP27, GGA or GGA derivatives induce an endoplasmic reticulum chaperone, BIP.

### Example 7

### Protection from Acetaminophen-Induced Hepatotoxicity In Vitro

In order to test the protective activity of GGA and GGA derivatives from acetaminophen-induced hepatotoxicity, a cytotoxicity assay is employed using human hepatoma (Bel-7402) cells in the presence of S9 mixture. Cell viability and mitochondrial permeability transition (MPT) is assessed in the presence or absence of GGA or GGA derivatives in combination with a cytotoxic concentration of acetaminophen. GGA or GGA derivatives show increased cell viability and protect from MPT disruption in the presence of acetaminophen compared with control conditions.

### Example 8

### Treatment of Non-Alcoholic Steatohepatitis

100 adults with nonalcoholic steatohepatitis are randomly assigned to receive GGA or a GGA derivative, each at a daily dose of about 10-200 mg, or placebo, for up to 12 months. The primary outcome is an improvement in histological features of nonalcoholic steatohepatitis. The extent of lobular inflammation, hepatocellular ballooning, and/or fibrosis is measured. The results are analyzed following methods well known in the art.

### Example 9

### Treatment of Non-Alcoholic Fatty Liver Disease (NAFLD)

A randomized, blinded, placebo-controlled study is performed on 100 patients with NAFLD diagnosed by ultrasound (US) and confirmed by liver biopsy (40 patients). The patients are randomized to receive GGA or a GGA derivative (each at a daily dose of 10-200 mg for up to 12 months) or placebo. All patients participate in an identical behavioral weight loss program, and undergo monthly evaluation by abdominal US. Liver enzyme levels, lipid profiles, insulin levels, and anthropometric parameters are also monitored, and all patients undergo nutritional follow-up evaluation. Patients also undergo a further liver biopsy examination as the study progresses. Serum alanine transaminase levels and steatosis by US are measured as non-limiting endpoints. The results are analyzed following methods well known in the art.

### Example 10

### GGA and GGA Derivative Activity in a Cardiac Ischemic Ischemia and Reperfusion In Vitro Model

GGA and GGA derivatives are tested for protective effects an in vitro ischemia/reperfusion cardiac disease model based on the contractile HL-1 cell line. Activity is assessed via apoptosis signaling, cell structure and energy-metabolism. The HL-1 cardiomyocytes (murine atrial tumor cell line) are maintained in monolayer culture with Claycomb-medium (Sigma, Germany), Heaving reached confluence and contractile activity, cells are maintained as subcultures. Induction of ischemia was carried out on vital cardiomyocytes at culture day four. The subconfluent, contractile HL-1 cardiomyocytes are placed in nutrient-deficiency medium containing 2.5 mM hydrogen peroxide solution in order to enhance the oxidative stress in HL-1 cells. In control cultures the medium exchange is carried out with standard supplemented Claycomb-medium. 8 h after ischemia induction samples are harvested and revitalization is induced in parallel by replacing nutrient-deficiency medium with fresh Claycomb-medium and incubating cells for another 16 hours. Cell proliferation analysis is done by flow cytometry. Apoptosis analysis is performed by terminal desoxynucleotidyl transferase-mediated dUTP nick end-labeling. Total number of cells are determined using 7-AAD nucleus staining. Additionally formaldehyde-fixed cells on glass coverslips are prepared for immuncytochemical staining. TUNEL assay is performed using an In Situ Cell Death Detection Kit, GGA and GGA derivatives reduce ischemia induced apoptosis and rescue ischemia-induced reduction of cell proliferation.

### Example 11

### GGA and GGA Derivatives Protect Against Myocardial Ischemia and Reperfusion Injury in Rats

Anesthetized male rats are treated once orally with GGA or a GGA derivative 24 h before ischemia, and subjected to ischemia for 30 min, followed by reperfusion for 4 h. Lactate dehydrogenase (LDH), creatine kinase (CK), malondialdehyde (MDA), superoxide dismutase (SOD) activity and infarct size are measured. The results show that pre-treatment with GGA or a GGA derivative significantly reduces the infarct size and the levels of LDH and CK after 4 h of reperfusion. GGA also significantly inhibits the increase in MDA levels and the decrease in SOD levels.

### Example 12

### Heat Shock Protein 70 Induced by GGA or GGA Derivative Protects Heterotopically Transplanted Hearts in Rats

A total of 20 donor rats are randomly divided into 2 groups. One of those receives an oral dose of GGA or a GGA derivative and one is a control group. Donor hearts are heterotopically transplanted into recipient rats 24 h after GGA administration. The levels of HSP70 expression in donor hearts and the variation of myocardial enzymes in receptor blood or donor hearts are measured 24 h after transplantation. The donated hearts are also examined under a microscope for pathological changes. HSP70 expression is increase in the GGA-treated group. Lactate dehydrogenase and creatine kinase muscle band concentrations in receptor blood are decreased in the GGA group compared to the control group. Moreover, the GGA group shows the lower malondialdehyde concentration and the higher atriphosphate concentration than the control group, demonstrated by the milder inflammatory injury in the transplanted hearts.

### Example 13

### Treatment of Cardiac Ischemia and Related Indications

A randomized, blinded, placebo-controlled study is performed on 100 patients diagnosed with cardiac ischemia, myocardial infarction or acute coronary syndrome based on coronary angiograms. The patients are randomized to receive GGA or a GGA derivative (each at a daily dose of 10-200 mg for up to 12 months) or placebo. GGA or a GGA derivative is directly administered, e.g., in an emergency room setting, to the coronary artery via a PCI/stent catheter followed by oral administration of GGA or a GGA derivative for several weeks. In some patients, administration of GGA or a GGA derivative occurs during percutanous intervention (PCI) while stenting through a catheter directly to the coronary artery and the site of infarction. Oral treatment follows preferably for at least 1 month following the heart attack. The incidence of angina is ascertained. The results are analyzed following methods well known in the art.

1. A method for treating inflammatory bowel disease and/or reducing one or more negative effects of inflammatory bowel disease comprising administering an effective amount of a composition of GGA or a GGA derivative to a subject in need thereof.

2. A method for treating chronic liver disease and/or reducing one or more negative effects of chronic liver disease comprising administering an effective amount of a composition of GGA or a GGA derivative to a subject in need thereof.

3. A method of treating a disorder selected from, acute liver injury from trauma, surgery or as a side effect of cancer treatment, acute liver failure caused by drug toxicity, cardiac ischemia, myocardial infarction, repurfusion injury and heart transplants, or a related disorder or condition, comprising administering a composition comprising an effective amount of geranylgeranyl acetone (GGA) or a GGA derivative, or a pharmaceutically acceptable salt thereof, and a pharmaceutically acceptable excipient, to a subject in need thereof.
